SEQUENCE LISTING (6 sequences)

SEQ ID NO: 1 - DNA, 21 bp, Artificial Sequence
  forward primer for CTX1 gene
agcaccagtaaagtgatggcc

SEQ ID NO: 2 - DNA, 21 bp, Artificial Sequence
  reverse primer for CTX1 gene
caaactctgcggaatctgacg

SEQ ID NO: 3 - DNA, 21 bp, Artificial Sequence
  forward primer for OXA8 gene
tttcgcaagaaataacccaaa

SEQ ID NO: 4 - DNA, 22 bp, Artificial Sequence
  reverse primer for OXA8 gene
tttagaatggtgatcgcatttt

SEQ ID NO: 5 - DNA, 21 bp, Artificial Sequence
  forward primer for TEM gene
gaccgaaggagctaaccgctt

SEQ ID NO: 6 - DNA, 22 bp, Artificial Sequence
  reverse primer for TEM gene
catagttgcctgactccccgtc

(19) United States
(12) Patent Application Publication
(10) Pub. No.: US 2008/0124769 A1
(43) Pub. Date: May 29, 2008

(54) APPARATUS AND METHOD FOR REDUCING NON-SPECIFIC AMPLIFICATION IN MULTIPLEX PCR

(75) Inventors: Sang-hyun PAEK, Seoul (KR); Jung-nam LEE, Seoul (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/936,321

(22) Filed: Nov. 7, 2007

(30) Foreign Application Priority Data
Nov. 7, 2006 (KR) 10-2006-0109526

(51) Int. Cl. C12P 19/34; C12M 1/00

(52) U.S. Cl. 435/91.2; 435/305.2

(57) ABSTRACT

Provided are a well plate for multiplex PCR including: a plurality of wells for multiplex PCR each well comprising at least two partitioned chambers formed by at least one barrier that extends in an upward direction from the bottom of the wells and has a lower height than the height of the wells, wherein the wells optionally comprise plugs that seal the partitioned chambers to be separated from each other, a multiplex PCR apparatus including the well plate, and a method of performing multiplex PCR using the multiplex PCR apparatus.

This application claims priority to Korean Patent Application No. 10-2006-0109526, filed on Nov. 7, 2006, in the Korean Intellectual Property Office, and all the benefits accruing therefrom under 35 U.S.C. §119, the disclosure of which is incorporated herein in its entirety by reference.

### BACKGROUND OF THE INVENTION

1. Field of the Invention

The present invention relates to a well plate. The well plate can be used for a multiplex polymerase chain reaction (PCR) to reduce non-specific amplification. The present invention further relates to a multiplex PCR apparatus comprising the well plate, and a method of performing a multiplex PCR using the multiplex PCR apparatus.

2. Description of the Related Art

Several conventional methods for amplifying a target nucleic acid sequence are known. For example, a target nucleic acid sequence can be amplified by a polymerase chain reaction (PCR), strand displacement amplification (SDA), transcription mediated amplification (TMA) that synthesizes target DNA from mRNA, nucleic acid sequence based amplification (NASBA), or the like. Generally, PCR methods provide the highest amplification efficiency, and are therefore the most widely used methods for amplifying a target nucleic acid sequence.

Recently, research directed to methods of performing DNA amplification in a small chip, such as a lab-on-a-chip or a small device, and analyzing DNA within a short period of time has been conducted. Common problems identified for DNA amplification methods in a small chip is that these methods often require that the genomes be purified from a sample, such as a blood sample or a tissue sample, or that the various target genes should be stably and maximally amplified in a small space such as a chip in order to obtain maximal information at a low cost. Multiple nucleic acid amplification products can be obtained simultaneously in a limited space using multiplex amplification methods, for example, multiplex PCR.

A multiplex PCR is a PCR method that produces at least two DNA amplification products from a single reaction solution. Multiplex PCR methods are performed using at least two primer pairs in the reaction solution. One known advantage of using a multiplex PCR is that multiple DNA amplification products can be obtained simultaneously in a limited space. However, problems arise which can effect amplification efficiency when a plurality of genes are amplified in a single reaction, as in a multiplex PCR, using a plurality of pairs of primers. For example, when a plurality of genes are amplified in multiplex PCR using a plurality of pairs of primers, PCR may not be performed efficiently due to factors such as primers competing against one another, the formation of primer dimers, amplification of non-specific PCR products, varied Tm value of each primer, and other design conditions that are independently taken into account for each primer. These factors can lead to reduced amplification efficiency or prevent amplification of some or all of the desired amplification targets.

In addition, several methods are known that reduce non-specific amplification in PCR. For example, U.S. Pat. No. 6,783,940 discloses a method of performing a polymerase chain reaction in the presence of an amount of sorbitol and dimethyl sulfoxide (DMSO) effective to increase yield of target molecules. When sorbitol and DMSO are used, non-specific amplification is significantly reduced compared with when they are not used.

U.S. Pat. No. 5,587,287 discloses a method of reducing non-specific amplification by reducing mispriming at a low temperature such that an inhibitor that inhibits DNA polymerase activity is bound to DNA at a temperature of 94° C. or less, but when temperature increases to 94° C. or above, the inhibitor does not inhibit DNA polymerase activity.

However, such methods described above have problems in that the reactions require the addition of specific materials to the PCR reaction. Further, the PCR conditions have to be adjusted when a primer sequence is modified, otherwise PCR products are not obtained at all.

In an attempt to develop an efficient method for multiplex PCR, the inventors found that non-specific amplification could be reduced in multiplex PCR by placing each primer pair of a plurality primers pairs into a separate partitioned chamber in a well to prevent amplification interference between pairs of primers, thus completing the present invention.

### SUMMARY OF THE INVENTION

In one embodiment, the invention provides a well plate comprising a plurality of wells, each well comprising at least two partitioned chambers formed by a barrier, wherein the barrier extends in an upward direction from the bottom of the well, wherein the height of the barrier is less than the height of the well; and optionally a removable plug for each well for sealing the partitioned chambers of the well such that a solution in a partitioned chamber is kept separated from solutions in other partitioned chambers within the well.

In another embodiment, the invention provides a multiplex PCR apparatus comprising the well plate.

In another embodiment, the invention provides a method of performing multiplex PCR comprising adding a PCR mixture comprising a nucleic acid template to each partitioned chamber of a well of a well plate, adding a primer pair to each partitioned chamber of the well of the well plate, wherein the primer pair is selected from a plurality of primer pairs, and wherein the well plate comprises a plurality of wells, each well comprising at least two partitioned chambers formed by a barrier, wherein the barrier extends in an upward direction from the bottom of the well, wherein the height of the barrier is less than the height of the well; and optionally a removable plug for each well for sealing the partitioned chambers of the well such that a solution in a partitioned chamber is kept separated from solutions in other partitioned chambers of the well.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a conventional 96 well plate;

FIG. 2 is a diagram illustrating a 96 well plate according to an embodiment of the invention;

FIG. 3 is a diagram illustrating an enlarged view of a portion of the 96 well plate according to an embodiment of the invention as used in PCR;

FIG. 4 is diagram illustrating an enlarged cross-sectional view of a portion of the 96 well plate according to an embodiment of the invention;

FIG. 5 is a schematic diagram illustrating a method of performing a multiplex PCR and purifying the product using an exemplary embodiment of the well plate;

FIG. 6 is an image showing results of an electrophoretic analysis of a single PCR in a single chamber, a multiplex PCR in a single chamber, and a multiplex PCR in a multiplex chamber according to an embodiment of the present invention; and

FIG. 7 is a graph illustrating the amount of DNA obtained for each amplicon in the experiment shown in FIG. 6

### DETAILED DESCRIPTION OF THE INVENTION

The invention will now be described more fully hereinafter with reference to the accompanying drawings, in which embodiments of the invention are shown. The invention may, however, be embodied in many different forms and should not be construed as limited to embodiments set forth herein. Rather these embodiments are provided so that this disclosure will be through and complete, and will fully convey the scope of the invention to those skilled in the art.

As used herein, the term “nucleic acid” means DNA or RNA, or a combination of both. The DNA or RNA can be in any possible configuration, i.e., in the form of double-stranded (ds) nucleic acid, or in the form of single-stranded (ss) nucleic acid, or as a combination thereof (in part ds or ss).

In one embodiment, the present invention provides a well plate comprising: a plurality of wells, each well comprising at least two partitioned chambers formed by at least one barrier, wherein the at least one barrier extends in an upward direction from the bottom of the wells, and wherein the height of the at least one barrier is less than the height of the wells, wherein the wells optionally comprise plugs that seal the partitioned chambers to be separated from each other. The well plate comprises at least two of the wells described above. The well plate is useful for various biological reactions in which multiplexing is desirable, for example, for multiplex PCR The well plate basically comprises a plurality of wells, each well comprising at least two partitioned chambers and plugs. The partitioned chambers are divided by at least one barrier. The barrier extends in an upward direction from the bottom of a well. The height of the barriers is less than the height of the well.

In one embodiment, the well plate optionally comprises plugs that seal the partitioned chambers to be separated from each other.

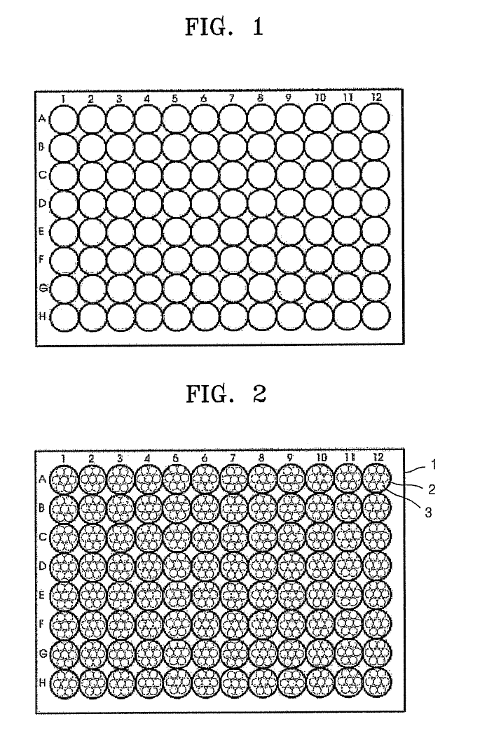

FIG. 1 is a diagram illustrating a conventional 96 well plate. When a multiplex PCR is conducted using a conventional 96 well plate as described in FIG. 1, a plurality of primer pairs are added to each well of the 96 well plate and then PCR is performed in a PCR device. One well recognized disadvantage with this method is that primer dimers, which do not correspond to the desired PCR products, can be produced, or, in some reactions the desired PCR products may not be produced. These problems occur because when a plurality of primer pairs exist inside one well that is used as a reactor one pair of primers can affect other pairs of primers.

FIG. 2 is a diagram illustrating a 96 well plate 1 according to an embodiment of the present invention. FIG. 2 illustrates a 96 well plate, but the present invention is not limited thereto, and the well plate of the present invention may include 8 wells, 12 wells, 384 wells, 1536 wells, or the like. As shown in FIG. 2., the 96 well plate 1, according to an embodiment of the invention, includes a plurality of partitioned chambers 3 in each of the 96 wells 2. In the conventional 96 well plate for a PCR illustrated in FIG. 1, a plurality of pairs of primers are added to one well. However, since each well of the well plate described in FIG. 2 comprises at least two partitioned chambers formed by at least one barrier, a primer pair can be added to each partitioned chamber 3 of a well 2. Therefore, each primer pair is separated from other primer pairs by the partitioned chambers 3. Accordingly, the primer pairs cannot affect one another in the PCR.

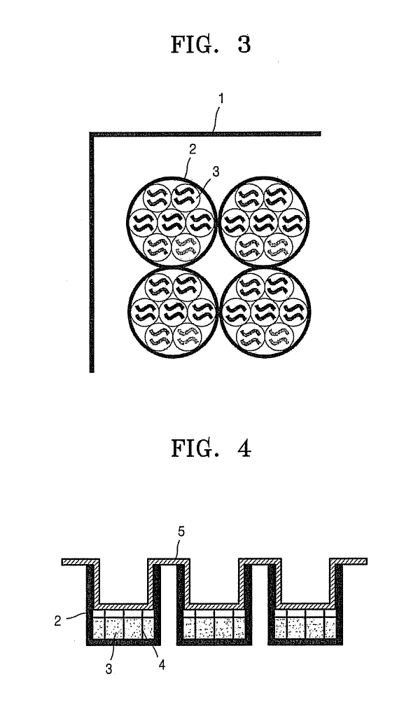

FIG. 3 is a diagram illustrating an enlarged view of a portion of the 96 well plate 1 for a PCR, according to an embodiment of the present invention. Referring to FIG. 3, it can be seen that an individual primer pair can be added to each partitioned chamber 3 of a well 2. Thus, a single primer pair can exist inside the partitioned chambers 3 in the wells 2. In another embodiment, a plurality of primer pairs can be added to a single partitioned chamber 3 of wells 2.

FIG. 4 is a diagram illustrating an enlarged cross-sectional view of a portion of the 96 well plate 1 for a PCR, according to an embodiment of the present invention. A barrier 4 extends in an upward direction from the bottom of each of the wells 2. As demonstrated in FIG. 4, the height of the barriers is less than that of the wells 2. Specifically, all of the barriers 4 in a given well have the same height; more specifically, all of the barriers 4 on the well plate have the same height. That is, in order to easily seal the partitioned chambers 3 with plugs 5, each chamber 3 may have the same height.

The barriers 4 and the wells 2 may be formed of a material comprising polypropylene, polystyrene, stainless steel, aluminum or the like, but are not limited thereto. However, the material used to form the barriers 4 and the wells 2 should be able to withstand high temperatures, including temperatures of up to about 95° C., up to about 100° C., or up to about 110° C., taking into account that the temperature of the chamber 3 for PCR increases by 95° C.

In one embodiment, the 96 well plate 1 optionally comprises removable plugs 5 that can be used to seal the partitioned chambers. Specifically, the plugs 5 cover top portions of the barriers 4 in the wells 2, and may further cover a portion of the inner sides of the wells 2 extending from the top portion of the barriers 4 to a top edge portion of the wells 2, the top edge portions of the wells 2 and any space between the top edge portions of adjacent wells 2. As illustrated in FIG. 4, the barriers 4 have a lower height than that of the wells 2. Therefore, in order for the plugs 5 to seal the partitioned chambers 3 by contacting the barriers 4, the plugs 5 have a concave shape concaving inwardly towards the wells 2. The depth by which the plugs 5 concave inwardly toward the wells 2 is determined by the height of the barriers 4. The greater the height of the barriers 4, the less the plugs 5 concave inwardly toward the wells 2.

The plugs 5 can be formed of a material such as rubber, silicone or the like, but are not limited thereto. However, the material used to form the plugs 5 should be able to withstand high temperatures, including temperatures of up to about 95° C., up to about 100° C., or up to about 110° C., taking account into that the temperature of the chamber 3 for a PCR increases by 95° C.

In one embodiment, in the 96 well plate 1, the number of the partitioned chambers 3 in each well 2 is a number selected from 2 to 50.

In some embodiments, the number of partitioned chambers 3 may be the same as the number of primer pairs added to each well 2. Further, the number of the partitioned chambers 3 in each well plate 1 may be the same as the number of primer pairs added to each well plate 1 for PCR. For example, when the number of primer pairs used in multiplex PCR is 5, there may be 5 partitioned chambers 3. That is, when the number of primer pairs is the same as the number of the partitioned chambers 3, each of the primer pairs can be independently placed in a different partitioned chamber 3.

In another embodiment, the number of the partitioned chambers 3 can be greater than or less than the number of primer pairs. When the number of the partitioned chambers 3 is greater than the number of primer pairs, the same number of partitioned chambers 3 as that of primer pairs is used, and multiplex PCR can be performed while the remaining partitioned chambers 3 remain empty. On the other hand, when the number of the partitioned chambers 3 is less than the number of primer pairs, at least two primer pairs, i.e., two primer pairs, three primer pairs, four primer pairs, etc., can be placed in one partitioned chamber 3. However, a plurality of primer pairs placed in the same partitioned chamber 3 are preferably pairs of primers that minimally affect PCR amplification. Therefore, it is not necessary that each primer pair be placed in a separate partitioned chamber 3, providing that a plurality of primer pairs placed in one partitioned chamber 3 do not affect PCR amplification of the targets. The selection of a plurality of primer pairs placed in the same partitioned chamber 3 can be determined by factors such as sequence homology between primer pairs, the length of primer pairs, and Tm. For example, the lower the sequence homology between primers of two or more primer pairs, the higher the probability that the primer pairs will not affect PCR amplification of the targets of the other primer pairs, thus allowing the primer pairs to be placed in the same partitioned chamber.

In another embodiment, the invention provides a multiplex PCR apparatus comprising the 96 well plate 1 illustrated in FIG. 2.

A PCR apparatus is an apparatus for performing PCR, and the multiplex PCR apparatus according to the current embodiment of the present invention comprises the well plate 1 illustrated in FIG. 2. In addition to the well plate 1, the multiplex PCR apparatus can further include a heater and a cooler. PCR apparatuses are commercially available from various suppliers, and the multiplex PCR apparatus according to the current embodiment of the present invention can be manufactured by installing the well plate used in the present invention in a conventional PCR apparatus.

In another embodiment, the invention provides a lab-on-a-chip comprising the multiplex PCR apparatus according to the present invention. Each of the functional elements of the multiplex PCR apparatus used in the present invention can be implemented as a process-on-a-chip using known microfluidic techniques and Micro Electro-Mechanical Systems (MEMS) devices, and furthermore, can be implemented as a lab-on-a-chip.

According to another embodiment, the invention provides a method of performing multiplex PCR using the multiplex PCR apparatus of the present invention, the method comprising adding a PCR mixture comprising a nucleic acid template and one or more primer pairs to each of a plurality of partitioned chambers.

According to the current embodiment, and unlike conventional methods of performing multiplex PCR, the method of performing multiplex PCR comprises adding a PCR mixture comprising a nucleic acid template to each partitioned chamber of a well of a well plate, adding a primer pair to each partitioned chamber of the well of the well plate, wherein the primer pair is selected from a plurality of primer pairs, and wherein the well plate comprises a plurality of wells, each well comprising at least two partitioned chambers formed by a barrier, wherein the barrier extends in an upward direction from the bottom of the well, wherein the height of the barrier is less than the height of the well; and optionally a removable plug for each well for sealing the partitioned chambers of the well such that a solution in a partitioned chamber is kept separated from solutions in other partitioned chambers of the well. This embodiment is in contrast to conventional methods of performing multiplex PCR in which a PCR mixture including a nucleic acid template and plurality of primer pairs are all added to a single chamber. The conventional method of performing multiplex PCR is well known in the art. When PCR is performed using the method of performing multiplex PCR according to an embodiment of the present invention, the production of non-specific PCR products is significantly reduced, and the desired PCR products can be obtained in good yield.

As used herein, the term “non-specific amplification” refers to amplification of a region of template nucleic acid that is not a region of the target nucleic acid. That is, non-specific amplification refers to amplification of a region of nucleic acid that is unrelated to the target nucleic acid sequence. The target refers to the portion of the nucleic acid template selected for specific amplification by a selected primer pair.

In embodiments of the present invention, primer pairs are added to a PCR reaction. Here, one primer of a pair of primers anneals to a positive strand (sense) of denatured template DNA, and the other primer of a pair of primers anneals to a negative strand (anti-sense) of denatured template DNA. Typically, the length of a primer is 12-25 nucleotides, but can be shorter or longer depending on the specific template sequence to be amplified. In general, oligonucleotide primers are chemically synthesized.

The oligonucleotide primers may be composed of adenosine, thymidine, guanosine, cytidine, uracil, nucleoside analogs (e.g., locked nucleic acids (LNA), peptide nucleic acid (PNA), phosphoramidites) and nucleosides containing or conjugated to chemical moieties such as radio-nuclides (e.g., ³²P, ³⁵S) and fluorescent molecules.

The enzyme that polymerizes the nucleotide triphosphates into the amplified fragments of the PCR may be any DNA polymerase, including heat-stable polymerases, known in the art. Polymerases that may be used in the present invention include, but are not limited to DNA polymerases isolated or derived from such organisms as Thermus aquaticus, Thermus thermophilus, Thermococcus litoralis, Bacillus stearothermophilus, Thermotoga maritime and Pyrococcus spp. The polymerase enzyme may be isolated from the bacteria or produced by recombinant DNA technology. Many suitable DNA polymerases are commercially available.

PCR reaction time, temperatures and cycle numbers may be varied to optimize a particular reaction. PCR reaction time and temperature are determined in three stages: denaturation, annealing and extension. One round of denaturation, annealing and extension is referred to as a “cycle.” Denaturation is generally conducted at a temperature that permits the strands of DNA to separate, typically at a temperature of 94-95° C. Denaturation of DNA is generally conducted for 1-30 seconds. During the annealing phase, oligonucleotide primers anneal to the target DNA in their regions of complementarity. Typically, in standard PCRs, the annealing temperature is 5-10° C. below the estimated Tm. The annealing phase is followed by an extension phase. PCR cycle numbers determine a desired amount of amplification. Preferably, the number of PCR cycle may be 20-30.

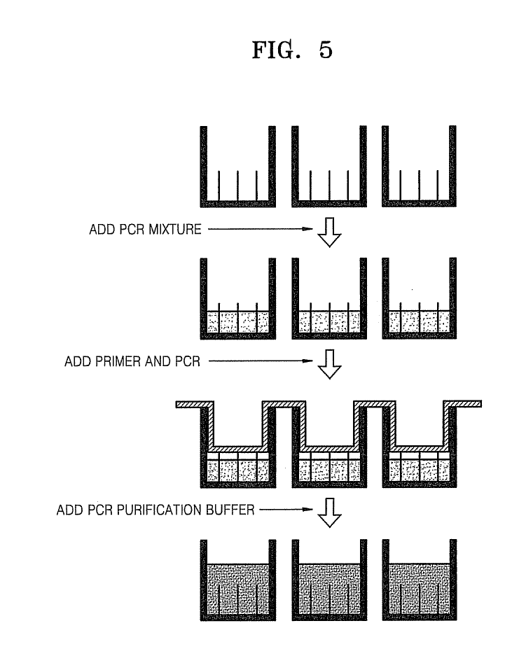

FIG. 5 is a schematic diagram illustrating a method of performing a multiplex PCR and purifying a product obtained by the multiplex PCR, according to an embodiment of the present invention. According to the method illustrated in FIG. 5, first, a PCR mixture is added to each of a plurality of partitioned chambers formed by barriers in a plurality of wells, then a primer pair from a plurality of primer pairs is added to each of the partitioned chambers. PCR is then performed under selected PCR conditions. After PCR is terminated, a PCR purification buffer is added to the chambers to recover PCR products. Following the addition of the PCR purification buffer to the well, the combined PCR mixture and PCR purification buffer can be removed from the plate well, wherein the amplified product can be purified or analyzed according to methods well known in the art.

In the method of performing a multiplex PCR according to the current embodiment of the present invention, each primer pair among the primer pairs for multiplex PCR can be placed in separate partitioned chambers. As described above, when each of primer pair is added to a separate partitioned chamber, interaction between primers can be prevented in the amplification reaction.

In one embodiment, the method of performing a multiplex PCR, the volume of the PCR mixture added to each partitioned chamber should be less than the volume of the partitioned chamber in order for the PCR mixture not to overflow from the partitioned chambers. Thus, as represented in FIG. 5, the height of the barriers can be higher than the height to which the partitioned chambers are filled with the PCR mixture.

In one embodiment, the PCR mixture used for the PCR reaction comprises template nucleic acid, dNTPs, Taq polymerase, a PCR buffer, and the like. A primer pair for addition to each partitioned chamber can be dissolved in any suitable buffer, for example, the PCR buffer. In another embodiment, the method of performing a multiplex PCR can further include purifying a PCR product obtained by the multiplex PCR. Purifying a PCR product can be accomplished by adding a PCR purification buffer to the partitioned chambers after the PCR is terminated. Preferably, when the PCR purification buffer is added, the depth of the solution of the PCR mixture and the PCR purification buffer becomes greater than the height of the barrier, and thus the solution of the PCR mixture and the PCR purification buffer in one partitioned chamber overflows into that of the other partitioned chambers. When a PCR mixture is initially added to the partitioned chambers prior to PCR, the PCR mixture should not overflow from the partitioned chambers. However, after PCR is terminated, the PCR purification buffer is added to the partitioned chambers so that the solution of the PCR mixture and the PCR purification buffer overflows into adjacent partitioned chambers. This is to permit PCR products to be conveniently recovered at once when the added PCR purification buffer has a larger volume than the volume of each of the partitioned chambers so as to overflow into the adjacent partitioned chambers. If the solution of the PCR mixture and the PCR purification buffer do not overflow into the adjacent partitioned chambers when a PCR purification buffer is added to the PCR mixture existing in a partitioned chamber, PCR products have to be individually recovered from each of the partitioned chambers. Therefore, the PCR purification buffer may be added to the partitioned chambers so that the solution of the PCR mixture and the PCR purification buffer overflows into the adjacent partitioned chambers.

The present invention will now be described in further detail with reference to the following Examples. These Examples are for illustrative purposes only and are not intended to limit the scope of the present invention.

### EXAMPLE 1

### PCR Efficiency of a Well Plate for Multiplex PCR According to an Embodiment of the Present Invention

For this example, PCR efficiency was determined using a well plate for multiplex PCR according to an embodiment of the present invention. PCR was performed using a GENEAMP PCR system 9700 (Applied Biosystems, USA). Unless otherwise specified, template DNA used in all the experiments was E. coli BL21 genomic DNA. Primer pair sequences used for PCR of amplification targets in the E. coli BL21 genomic DNA are shown in Table 1 below:

TABLE 1

| | Forward | Reverse |
|---|---|---|
| gene | sequence (5′→3′) | sequence (5′→3′) |
| CTX1 | AGCACCAGTAAAGTGATGGCC | CAAACTCTGCGGAATCTGACG |
| | (SEQ ID NO:1) | (SEQ ID NO:2) |
| OXA8 | TTTCGCAAGAAATAACCCAAA | TTTAGAATGGTGATCGCATTTT |
| | (SEQ ID NO:3) | (SEQ ID NO:4) |
| TEM | GACCGAAGGAGCTAACCGCTT | CATAGTTGCCTGACTCCCCGTC |
| | (SEQ ID NO:5) | (SEQ ID NO:6) |

Unless otherwise specified, a PCR mixture including 1×PCR buffer (Solgent Co. Ltd.), 200 μM of dNTP, 0.2 μM of each primer, 5U of Taq DNA polymerase, the mixture having a total volume of 50 μl. PCR temperature conditions are as follows: initial denaturation at 95° C. for 1 minute, 31 cycles of 95° C. for 5 seconds, 62° C. for 13 seconds and 72° C. for 15 seconds, and then final extension at 72° C. for 1 minute. Quantitative analysis for the PCR products obtained was conducted using a LABCHIP for the Agilent Technologies 2100 Bioanalyzer.

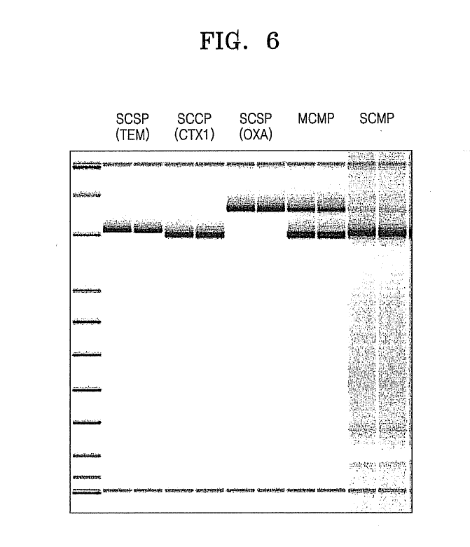

FIG. 6 is an image showing results of performing a single PCR in a single chamber using the TEM, CTX1 or OXA primer pairs; a multiplex PCR in a single chamber using the TEM, CTX1 and OXA primer pairs; and a multiplex PCR in a multiplex chamber using the TEM, CTX1 and OXA primer pairs according to an embodiment of the present invention.

As can be seen in FIG. 6, in the case of single PCR in a single chamber (SCSP), TEM, CTX1 and OXA genes each show one product amplified in its amplicon size. In the case of multiplex PCR in a conventional single chamber (SCMP), it can be seen that yield of the amplicon from the OXA gene is significantly decreased. However, in the case of performing multiplex PCR in a multiplex chamber (MCMP) using a well plate according to an embodiment of the present invention, it can be seen that the amplified OXA product having the same size as in SCSP and the same yield of amplicon as in SCSP can be obtained.

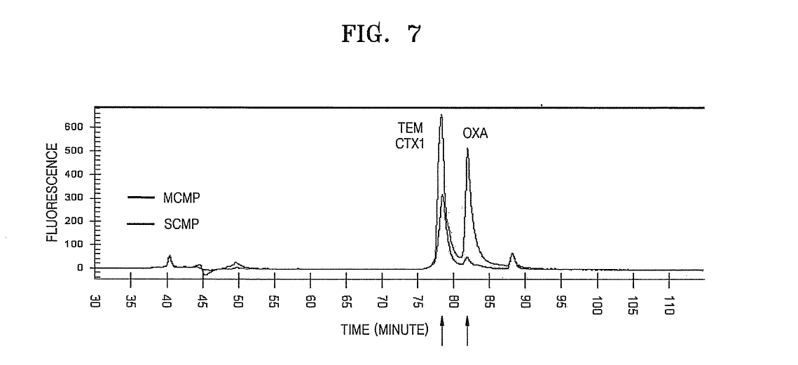

FIG. 7 is a graph illustrating the amount of DNA obtained in the experiment shown in FIG. 6 for each amplicon. In FIG. 7, two arrows represent the positions of PCR products of TEM, CTX1, and OXA. As can be seen in FIG. 7, in the case of SCMP, the amplicon of the OXA gene, amplified using the OXA primer pair, is barely produced while non-specific amplicon bands are produced. On the other hand, for the case of MCMP, only the desired amplicon band is obtained. Therefore, when multiplex PCR is performed using a well plate according to an embodiment of the present invention, the production of non-specific PCR products, which is generally observed in multiplex PCR, can be significantly reduced, and also desired PCR products can be obtained.

According to the present invention, a plurality of PCR reactions are each conducted in a different partitioned chamber due to the structure of the multiplex PCR apparatus using the well plate according to the present invention. By conducting amplification methods, such as a multiplex PCR reaction, using the well plate according to an embodiment of the present invention reduces the production of non-specific products that are produced by non-specific binding, and only a target DNA can be amplified. In addition, even under a condition of high homology between primers, mispriming is reduced or primer dimers are not produced. Accordingly, the time-consuming and labor-intensive process of checking for reactions between primers required for conventional multiplex PCR is not required.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A well plate comprising:
a plurality of wells, each well comprising at least two partitioned chambers formed by a barrier,
wherein the barrier extends in an upward direction from the bottom of the well,
wherein the height of the barrier is less than the height of the well; and
optionally a removable plug for each well for sealing the partitioned chambers of the well such that a solution in a partitioned chamber is kept separated from solutions in other partitioned chambers within the well.

2. The well plate of claim 1, wherein the number of wells is 8, 12, 96, 384, or 1,536.

3. The well plate of claim 1, wherein the partitioned chambers in a well are formed by multiple barriers and the barriers have the same height.

4. The well plate of claim 1, wherein the plug seals the plurality of wells and covers
a top portion of the barrier in each well,
a portion of the inner sides of each well extending from the top portion of the barrier to a top edge portion of the well,
the top edge portion of each well, and
a space between the top edge portions of adjacent wells.

5. The well plate of claim 1, wherein the number of partitioned chambers in each well is a number selected from 2 to 50.

6. The well plate of claim 1, wherein the barrier and the well are formed of a material comprising polypropylene, polystyrene, stainless steel, or aluminium.

7. The well plate of claim 1, wherein the plug is formed of rubber or silicone.

8. A multiplex PCR apparatus comprising the well plate according to claim 1.

9. A lab-on-a-chip comprising the multiplex PCR apparatus according to claim 8.

10. A method of performing multiplex PCR, comprising
adding a PCR mixture comprising a nucleic acid template to each partitioned chamber of a well of a well plate,
adding a primer pair to each partitioned chamber of the well of the well plate,
wherein the primer pair is selected from a plurality of primer pairs, and
wherein the well plate comprises
a plurality of wells, each well comprising at least two partitioned chambers formed by a barrier,
wherein the barrier extends in an upward direction from the bottom of the well,
wherein the height of the barrier is less than the height of the well; and
optionally a removable plug for each well for sealing the partitioned chambers of the well such that a solution in a partitioned chamber is kept separated from solutions in other partitioned chambers of the well.

11. The method of claim 10, wherein each primer pair of the plurality of primer pairs is placed in a separate partitioned chamber of the well.

12. The method of claim 10, wherein the height of the barrier is greater than the height to which the partitioned chamber is filled with the PCR mixture.

13. The method of claim 10, further comprising
adding a PCR purification buffer to the partitioned chambers of the well after PCR is terminated; and
purifying a PCR product from the partitioned chambers.

14. The method of claim 13, wherein the amount of PCR purification buffer added to the partitioned chambers is such that the depth of the solution in the partitioned chambers becomes greater than the height of the barriers between each of the partitioned chambers.
